(19) United States
(12) Patent Application Publication
(10) Pub. No.: US 2021/0030761 A1
(43) Pub. Date: Feb. 4, 2021

(54) TREATMENT AND PREVENTION OF ALPHA HERPES VIRUS INFECTION

(71) Applicant: THE REGENTS OF THE UNIVERSITY OF COLORADO, a body corporate, Denver, CO (US)

(72) Inventors: Maria A. NAGEL, Aurora, CO (US); Andrew N. BUBAK, Aurora, CO (US)

(21) Appl. No.: 17/042,623

(22) PCT Filed: Mar. 28, 2019
(86) PCT No.: PCT/US19/24521

§ 371 Date: Sep. 28, 2020

Related U.S. Application Data
(60) Provisional application No. 62/649,564, filed on Mar. 28, 2018.

(51) Int. Cl. A61K 31/5377; A61K 31/675; A61K 31/4545; A61K 31/438; A61P 31/22

(52) U.S. Cl. CPC A61K 31/5377; A61K 31/675; A61K 45/06; A61K 31/438; A61P 31/22; A61K 31/4545

(57) ABSTRACT

Methods for decreasing or inhibiting herpesviridae (HV) infection, or pathogenesis of a cell, or a symptom, or pathology associated with a herpesviridae (HV) infection or pathogenesis or an adverse side effect of herpesviridae (HV) infection or pathogenesis in vitro, ex vivo, or in vivo. These methods include treating a subject with an NK-1 receptor antagonist.

### GOVERNMENT INTEREST

This invention was made with government support under grant numbers NS094758, AG032958, and NS093716, awarded by National Institutes of Health. The United States government has certain rights in the invention.

### TECHNICAL FIELD

The invention relates to methods for decreasing or inhibiting herpesviridae infection or a symptom or pathology associated with a herpesviridae infection by treating a subject with an NK-1 receptor (NK-1R) antagonist.

### BACKGROUND

Herpesviruses represent a family of DNA viruses that include herpes simplex virus (HSV) 1 and 2, varicella zoster virus (VZV), Epstein-Barr virus (EBV), cytomegalovirus (CMV), human herpesvirus (HHV) 6 and 7 and Kaposi sarcoma—associated herpesvirus (HHV-8). These viruses have several common exclusive characteristics: primary infections usually occur during infancy or childhood; all herpesviruses become latent following primary infection, residing in the host nervous or hematopoietic systems. Latency is identified by serum antibodies and the antibody positivity rate among adults (seroprevalence) of 65% for HSV1 and 54% (by age 40) for CMV in the United States and reflects the acquisition rates of these viruses in childhood. During latency, reactivations are common and mostly asymptomatic but result in virus shedding in mucus membranes, which is the source of infection for contacts of asymptomatic shedders. Infections primarily acquired in adulthood tend to be more severe. Symptomatic reactivations are rare among immunocompetent people except for VZV reactivations (zoster or shingles), but are very common among immunocompromised patients.

Varicella zoster virus (VZV) is an exclusively human neurotropic virus that produces varicella on primary infection, after which it establishes latency in ganglionic neurons within cranial nerve, dorsal root, sympathetic, parasympathetic and enteric ganglia along the entire neuraxis, as well as in adrenal glands. With age or immunosuppression, VZV reactivates from one or more ganglia and typically travels peripherally to skin to produce zoster in the corresponding dermatome. However, virus can also travel centrally along nerve fibers to infect the spinal cord and produce myelitis with or without zoster rash.

VZV myelitis is one of several neurological complications of VZV reactivation and presents as a self-limiting paraparesis with or without sensory features and sphincter problems. In immunocompromised individuals, VZV myelopathy can be progressive and fatal with frank virus infection of the spinal cord. Pathogenic mechanisms of VZV myelitis are not well-characterized, mainly because VZV is an exclusively human virus and there are no animal models to study neurological complications of VZV infection. However, postmortem analyses of 8 patients with VZV myelopathy revealed that astrocytes, as well as oligodendrocytes and neurons, in the spinal cord were infected horizontally and longitudinally. The site of cord involvement was associated with VZV reactivation and zoster rash, supporting the notion that after VZV reactivation from dorsal root ganglionic neurons, virus travels peripherally to skin to produce zoster and centrally to the spinal cord to produce myelitis along the same dermatome(s). Additional support that VZV myelitis can be caused by direct VZV invasion of the spinal cord is provided by cases of acute and rapidly progressive, virologically-verified VZV myelitis in patients with acquired immunodeficiency syndrome, who improved with aggressive antiviral therapy, as well as cases of acute VZV myelitis in multiple sclerosis patients on immunomodulatory therapy, who also improved upon antiviral therapy.

Of the multiple cell types within the spinal cord, astrocytes are most likely a key contributor to the central nervous system (CNS) spread of infection and the development of VZV myelitis, because astrocytes are the most abundant glial cell type in the CNS with a high capacity for migration and are crucial regulators of neuroinflammation. Current knowledge of VZV infection of astrocytes is limited to brain astrocytes. Postmortem immunohistochemical studies of brain from VZV encephalitis cases showed that brain astrocytes are infected with VZV and are preferentially infected over other cell types. In vitro studies showed that astrocytes isolated from human brain are permissive to VZV infection, with VZV downregulating expression of glial fibrillary protein in these cells.

The most common anti-herpesvirus agents are nucleoside analogues, including acyclovir (an inactive drug that becomes activated in cells infected with HSV or VZV and eventually inhibits viral DNA). Other nucleoside analogues include valacyclovir, brivudin, cidofovir penciclovir, famciclovir, ganciclovir, valganciclovir, idoxuridine, trifluridine and vidarabine. These agents possess variable activity for different herpesviruses notably differing in their anti-CMV activity. Notable toxicities of nucleoside analogues myelosuppression, neurotoxicity, and nephrotoxicity.

Foscarnet, a pyrophosphate analogue, leads to reversible and incomplete DNA polymerase inhibition with good anti-viral activity. But foscarnet, has a poor toxicity profile with a need for intense electrolyte monitoring and renal failure, and produces painful genital ulcers and hemorrhagic cystitis in transplanted patients. Foscarnet is commonly used as a second-line treatment, following resistance or intolerance to nucleoside analogues.

Thus, there is a need for alternative effective agents for herpesviridae (HV) infection or pathogenesis with better toxicity profile than currently available therapies.

### SUMMARY

The present inventors have tested whether primary human astrocytes isolated from spinal cord are permissive to VZV infection, providing an in vitro model to study VZV myelitis, VZV myelopathy, VZV neuropathy, VZV pancreatitis (including VZV-associated diabetic complications), VZV keratitis, and VZV vasculopathy pathogenesis. They also tested whether blockade of the neurokinin-1 receptor (NK-1R), which is involved in cytoskeletal rearrangements and formation of cellular processes, including lamellipodia and filopodia, that reportedly facilitate VZV spread, inhibits these VZV-induced cellular extensions and subsequent viral spread in spinal astrocytes. The inventors have surprisingly discovered that neurokinin-1 receptor (NK-1R) antagonists are effective as antiviral drugs against VZV.

NK-1R antagonists reduced VZV infection in the following primary human cell types: Spinal astrocytes (associated with VZV myelitis, VZV myelopathy; can present as paralysis, and incontinence); Perineurial cells (associated with VZV neuropathy, provides a route of virus entry to the PNS and CNS, as well as a route of virus exit allowing infection of multiple organs during reactivation); Pancreatic islet cells (associated with VZV pancreatitis and VZV-associated diabetic complications); Keratocytes (associated with VZV keratitis, and presents as vision loss); Brain vascular adventitial fibroblasts (associated with VZV vasculopathy, and presents as stroke, hemorrhage, aneurysm, dissection, venous thrombosis). Additionally, the inventors demonstrated that NK-1R antagonists reduces VZV infection in Guinea pig lung fibroblasts. This provides evidence that VZV can infect guinea pig cells, thereby serving as a small animal model of VZV infections, and demonstrating that the therapeutic effect of the NK-1R antagonists is conserved across highly divergent species.

Thus, the inventors have now invented a novel treatment which is efficacious in treating and preventing VZV infection and spread, and pathogenic complications of VZV infection and reactivation.

This disclosure provides methods of treating or preventing infection and spread of herpesviridae (HV). These methods include providing a subject with protection against a HV infection or pathogenesis, by administering a sufficient amount of an NK-1R antagonist to provide the subject with protection against HV infection or pathogenesis.

These methods also include treating a subject in need of treatment for HV infection or pathogenesis, by administering a sufficient amount of an NK-1R antagonist to treat the subject for the herpesviridae (HV) infection or pathogenesis.

These methods also include decreasing susceptibility or inhibiting HV reactivation from latency in a subject, comprising administering a sufficient amount of an NK-1R antagonist to decrease susceptibility or inhibit HV reactivation from latency in the subject.

In these methods, the NK-1R antagonist may be aprepitant, rolapitant, fosaprepitant, lanepitant, befetupitant, or combinations thereof.

In these methods, the NK-1R antagonist may be administered prior to, concurrently with, or following infection of the subject with HV, exposure to or contact of the subject with HV, or reactivation of HV or following development of a symptom or pathology of acute or chronic HV infection, or reactivation of HV from latency.

In these methods, the HV may be an alpha-herpesvirus, beta-herpesvirus, or gamma-herpesvirus. The alpha herpes virus may be herpes simplex virus-1 (HSV-1), herpes simplex virus-2 (HSV-2), or varicella zoster virus (VZV/HHV-3). In these methods, the HV infection may be in a latent state, active state, or reactivated state.

In these methods, the subject may be provided with partial or complete protection against HV infection or pathogenesis, a symptom or pathology caused by HV infection, or pathogenesis or reactivation of HV from latency.

In these methods, the therapy may reduce, decrease, inhibit, ameliorate, or prevent onset, severity, duration, progression, frequency, or probability of one or more symptoms or pathologies associated with or caused by HV infection or pathogenesis, or reactivation of HV from latency, in the subject.

In these methods, the therapy may prevent or inhibit a worsening or progression of HV infection or one or more symptoms or pathologies associated with HV infection, or pathogenesis or reactivation of HV from latency.

In these methods, the symptoms or pathologies treated or prevented may include myelitis, myelopathy, neuropathy, pancreatitis, VZV-associated diabetic complications, vasculopathy, lesions, ulcers, canker sore, close sore, rash, boils, gingivostomatitis, herpes gladiatorum, eczema herpeticum, swollen lymph nodes, pneumonitis, pneumonia, hepatitis, meningitis, encephalitis, keratitis, genital herpes, esophagitis, hemiparesis, shingles, chicken pox, mononucleosis, chronic or acute pelvic inflammatory disease (PID), proctitis, colitis, and/or nerve damage.

In these methods, the therapy may stabilize the HV infection or one or more symptoms or pathologies associated with the HV infection or pathogenesis or reactivation of HV from latency.

In these methods, the therapy may reduce or decrease HV titer, viral load, viral replication, viral proliferation or a viral protein, or inhibit or prevents increases in HV titer, viral load, viral replication, viral proliferation, or a viral protein.

In these methods, the therapy may reduce or decrease susceptibility of the subject to HV infection or one or more symptoms or pathologies associated with HV infection or pathogenesis or reactivation of HV from latency.

In these methods, the subject may be immunocompromised, or may be a candidate for, or has received an immunosuppressant treatment, or is a candidate for or has received a tissue or organ transplant.

These methods may further comprise administering to the subject an additional HV treatment, such as an additional agent effective for treatment of HV, or an agent to treat or reduce a side effect of an HV treatment. For example, additional treatments may include a protease inhibitor, a reverse transcriptase inhibitor, a virus fusion inhibitor, or a virus entry inhibitor.

This Summary is neither intended nor should it be construed as representative of the full extent and scope of the present invention. Moreover, references made herein to “the present invention,” or aspects thereof, should be understood to mean certain embodiments of the present invention and should not necessarily be construed as limiting all embodiments to a particular description. The present invention is set forth in various levels of detail in this Summary as well as in the attached figures and the Detailed Description and no limitation as to the scope of the present invention is intended by either the inclusion or non-inclusion of elements, components, etc. in this Summary. Additional aspects of the present invention will become more readily apparent from the Detailed Description, particularly when taken together with the drawings.

### BRIEF DESCRIPTION OF DRAWINGS

FIGS. 1A-1D shows infection of primary human spinal astrocytes (HA-sp) with VZV. Quiescent HA-sp were mock- or VZV-infected and analyzed by immunofluorescence at 3 days post-infection using antibodies against glial fibrillary protein (GFAP, astrocyte marker) or VZV glycoprotein B (gB). FIG. 1A shows that GFAP was seen in all cells (light grey) confirming a pure astrocyte culture. FIG. 1B shows a cell expressing VZV gB; note the processes (lamellipodia) of the infected cell extending to surrounding uninfected cells, demonstrating that astrocytes were permissive to VZV infection. FIG. 1C is a higher magnification image of a VZV-infected astrocyte showing strikingly long lamellipodia containing filopodia (short arrows) that extends towards an uninfected cell; filopodia were also seen sprouting from the cell body (long arrows). FIG. 1D is a phase-contrast image overlay demonstrating normal morphology of uninfected bystander astrocytes (clear) and 2 lamellipodia arising from a VZV-infected astrocyte. Magnification was 400× in FIGS. 1A, 1B, 1D, and 600× in FIG. 1C.

FIG. 2A shows neurokinin-1 receptor (NK-1R) localizes to the nucleus in VZV-infected primary human spinal astrocytes (HA-sp). Quiescent HA-sp were mock- or VZV-infected and analyzed by immunocytochemistry at 3 days post-infection using antibodies against NK-1R and VZV glycoprotein E (gE). NK-1R is expressed diffusely in the cytoplasm of mock-infected cells (panel A) and predominantly outside of the nuclear membrane as visualized by a nuclear z-stack (panel B). In contrast, NK-1R is expressed predominantly in the nucleus of VZV-infected cells (panel C) with VZV gE in cytoplasm (panel C); a nuclear z-stack of a VZV-infected astrocyte confirms the presence of NK-1R within the nucleus (panel D).

FIG. 2B shows Neurokinin-1 receptor translocating to the nucleus in VZV-infected adult primary human perineurial cells (HPNCs) (top panels compared to bottom panels).

FIG. 2C shows the Neurokinin-1 receptor is upregulated in VZV-infected (lower panels) primary adult human pancreatic islet cells (identified by insulin staining; “INS”) compared to mock infected cells (upper panels).

FIG. 2D shows Neurokinin-1 receptor translocates to the nucleus (arrow in right panel) in VZV-infected adult primary human keratocytes but not in mock-infected cells (left panel).

FIG. 2E shows Neurokinin-1 receptor translocates to the nucleus (arrow in right panel) in VZV-infected guinea pig lung fibroblasts compared to vehicle-treated cells (left panel).

FIGS. 3A-3C demonstrate that substance P application does not induce nuclear localization of the neurokinin-1 receptor (NK-1R). In quiescent primary human spinal astrocytes treated with 10⁻⁶ M substance P for 60 minutes, NK-1R is not seen in the nucleus but is instead seen in the cytoplasm, predominantly around the nucleus as expected (FIG. 3A, light grey) which is confirmed by a nuclear z-stack (FIG. 3B, light grey). Additionally, substance P treatment failed to induce elongated cell processes; astrocytes retained their polygonal morphology as visualized by CellTracker Deep Red (Invitrogen) in FIG. 3C, light grey). Magnification for FIGS. 3A and 3C was 400× and 600× for FIG. 3B.

FIG. 4 demonstrates that aprepitant, a neurokinin-1 receptor (NK-1R) antagonist, inhibits cellular processes in VZV-infected primary human spinal astrocytes (HA-sp). Quiescent HA-sp were VZV-infected in the presence of vehicle with or without 10 aprepitant and analyzed by immunocytochemistry at 3 days post-infection using antibodies against NK-1R and VZV glycoprotein B (gB). Panel A shows VZV-infected astrocytes expressing VZV gB had long, extended processes (lamellipodia) and predominantly nuclear localization of NK-1R. High magnification in panel B shows a network of elongated processes extending from cell bodies of VZV-infected astrocytes (arrows); note the nuclear localization of NK-1R. Panel C demonstrates that application of the NK-1R antagonist aprepitant reduces the extension of processes in infected cells expressing VZV gB, with fewer cells containing nuclear NK-1R. High magnification in panel D demonstrates a clear reduction in lamellipodia of infected cells, but retention of nuclear NK-1R in some cells. Magnification was 200× for panels A, and C, and 400× for panels B and D.

FIGS. 5A-5H show aprepitant treatment of cells with established VZV infection reduces viral DNA and spread. Quiescent spinal astrocytes were VZV-infected. FIG. 5A depicts a timeline of infection and treatment. At 12 hours post-infection (HPI), spinal astrocytes were treated with vehicle or with 10 μM aprepitant, a neurokinin-1 receptor (NK-1R) antagonist. Treatment was reapplied at 24 and 48 HPI. At 12, 24, 48, and 72 HPI, cells were visualized by light microscopy in untreated and treated cultures and harvested for DNA. As shown in FIG. 5B, compared to untreated VZV-infected cells, aprepitant-treated VZV-infected cells had significantly lower amounts of VZV DNA at 48 (121±29.2 versus 10.5±3.8; p<0.01, mean±SD) and 72 HPI (452.9±122.9 versus 24.9±6.9; p<0.01, mean±SD) as quantified by qPCR [y-axis represents fold-change of VZV-infected treated with vehicle only (dotted line; n=3) or aprepitant (solid line; n=3)]. Corresponding light microscopy images demonstrate the formation of a cytopathic effect at 48 and 72 HPI in VZV-infected vehicle-only samples (FIG. 5C, arrows in upper panels) but not in aprepitant-treated samples (FIG. 5C, lower panels). FIG. 5D shows that aprepitant and rolapitant significantly reduced total VZV DNA at 72 hours post-infection (HPI), in HPNC cells treated with either NK-1R antagonist when treated at time of infection as well as at 24 HPI (*, P<0.05; **, P<0.01; ***, P<0.001). FIG. 5E shows significantly reduced total VZV DNA in human pancreatic islet cells at 6 days post-infection compared to vehicle-treated islets following treatment with aprepitant (7.5 μM) (*, P<0.05; **, P<0.01; ***, P<0.001). FIG. 5F shows significantly reduced total VZV DNA in human keratocytes treated with aprepitant (7.5 μM) at 4- and 7- days post-infection (DPI) compared to vehicle-treated cells (*, P<0.05; **, P<0.01; ***, P<0.001). FIG. 5G shows a dose-dependent reduction in total VZV DNA in primary human brain vascular adventitial fibroblasts infected with VZV and treated with aprepitant (5.0 μM and 7.5 μM) (*, P<0.05; **, P<0.01; ***, P<0.001). FIG. 5H shows significantly reduced total VZV DNA at 72 hours post-infection in Guinea Pig Lung Fibroblasts treated with aprepitant (7.5 μM) compared to vehicle-treated cells (*, P<0.05; **, P<0.01; ***, P<0.001). FIG. SI shows significantly reduced total VZV DNA post-infection in primary human microglia treated with aprepitant (7.5 μM) compared to vehicle-treated cells (*, P<0.05).

### DETAILED DESCRIPTION

The inventors used primary human spinal astrocytes to study VZV infection in the context of VZV spinal cord disease and identified neurokinin-1 receptor (NK-1R) antagonists as a novel antiviral drug against VZV. Several important points emerged from this study.

Understanding the mechanisms of VZV infection of spinal astrocytes that lead to clinical disease is essential, because astrocytes are the most abundant glial cell type of the CNS and have a high capacity for migration that may facilitate virus spread. Moreover, astrocytes are key regulators of neuroinflammation and can contribute to CNS disease through alterations in its normal function. Prior studies of VZV infection of human astrocytes were limited to immunohistochemical studies of brain from VZV encephalitis patients and from isolated brain astrocytes in vitro, which showed that these cells were preferentially permissive to infection over other cell types and able to downregulate GFAP. The inventors have confirmed the identity of their experimental cells as spinal astrocytes based on GFAP expression and showed that VZV-infected spinal astrocytes formed strikingly elongated cell processes (lamellipodia), with filopodia extending along these processes and around the cell body. While outgrowth of these processes resembles morphological changes seen in astrocyte activation and gliosis in response to CNS injury, these VZV-induced spinal astrocyte processes were not diffusely spread but rather appeared to migrate preferentially towards uninfected cells; processes do not extend to areas absent of uninfected cells. This unusual morphology in VZV-infected spinal astrocytes, as well as nuclear localization of NK-1R in the absence of endogenous ligand substance P, was not seen in adjacent uninfected bystander cells, indicating that direct virus infection was required for these alterations; this morphology and NK-1R nuclear localization was also not seen in uninfected cells treated with substance P. Furthermore, the expected reactive gliosis of surrounding uninfected cells exposed to VZV-infected cells and cytokines, chemokines and other soluble factors presumably released did not occur, with no signs of activation and with normal maintenance of their polygonal shapes in vitro, suggesting that the “danger” signals to prime surrounding astrocyte reactivity were not present or functional. The induction of extensive lamellipodia and filopodia in VZV-infected spinal astrocytes are most likely involved in cell-to-cell spread of VZV, consistent with a study showing that during VZV infection of human melanoma (MeWo) cells, VZV particles emerged on the cell surface amid actin-based filopodia, which were abundant within viral highways.

Further support for the role of VZV-induced, nuclear NK-1R mediated lamellipodia and filopodia formation in viral spread comes from the pharmacological blockade of such extension formations by aprepitant, an NK-1R antagonist. In spinal astrocytes with already established VZV infection, addition of aprepitant prevented formation of these cellular extensions and viral spread, as well as blocked significant increases in viral DNA. The rationale for targeting the NK-1R signaling pathway in VZV infection using NK-1R antagonists includes the following: (1) substance P ligand binding to NK-1R mediates the interaction between the immune system and nervous system; (2) NK-1R modulates cytoskeletal rearrangements, membrane blebbing and filopodia formation, which are important in VZV infection; (3) NK-1R signaling has been shown to be involved in RNA virus infection, e.g., substance P in plasma of HIV-infected individuals is elevated, and addition of substance P in vitro enhances HIV replication in blood-derived monocyte-derived macrophages; (4) NK-1R plays a role in measles virus spread in neurons, and NK-1R antagonists inhibit this effect; and (5) aprepitant is currently a safe and commercially available oral and intravenous drug that crosses the blood-brain barrier and is used as an anti-emetic agent for patients undergoing chemotherapy.

While the antiviral effects of aprepitant in HIV have been demonstrated by interference with substance P ligand binding to NK-1R, the mechanism in VZV infection is unclear, the inventors' analysis shows that VZV-infected spinal astrocytes do not contain substance P and that NK-1R translocates to the nucleus. Typically, NK-1R is expressed on the astrocyte cell surface and when its ligand, substance P binds, the ligand-receptor complex is internalized predominantly to the perinuclear region in the cytoplasm and not within the nucleus. Activation of this receptor at the cellular membrane results in the stimulation of adenylyl cyclase and the production of plasma membrane cAMP, as well as additional G protein-dependent endosomal signaling once internalized. During VZV infection, there is aberrant localization of NK-1R into the nucleus in the absence of detectable substance P by ELISA. Thus, it is possible that a substance P-like molecule is present in VZV-infected cells that contributes to aberrant nuclear NK-1R localization. Specifically, VZV glycoprotein B shares significant sequence homology to the active binding site of substance P, although its ability to bind to NK-1R remains to be determined. Thus, the inventors identified a novel, substance P-independent, proviral function of nuclear NK-1R associated with lamellipodia formation and viral spread that is distinct from substance P-induced NK-1R cell membrane/cytoplasmic localization without lamellipodia formation. These results show that binding a putative viral ligand to NK-1R produces a dramatically different NK-1R downstream effect than binding of substance P.

Overall, the inventors have provided an in vitro model to study infection of primary human spinal astrocytes in the context of VZV spinal cord disease. In addition, they have identified a novel pro-viral function of nuclear NK-1R in the absence of its endogenous ligand substance P that results in formation of lamellipodia and filopodia, which provide “highways” for cell-to-cell spread of VZV. The description of VZV-exploited NK-1R function in this study is critical to the knowledge of VZV pathogenesis in the central nervous system. Targeting NK-1R with an antagonist provides an antiviral therapy against VZV and a much-needed alternative/adjuvant to treatment of recurrent or disseminated VZV infections, because the currently available drugs (acyclovir, valacyclovir, famacyclovir) all share the same mechanism of action as nucleoside analog inhibitors of viral DNA replication.

In accordance with the inventors' results, this disclosure provides methods for decreasing or inhibiting herpesviridae (HV) infection or pathogenesis, or a symptom or pathology associated with a herpesviridae (HV) infection or pathogenesis, or an adverse side effect of herpesviridae (HV) infection or pathogenesis, in vitro, ex vivo, or in vivo. Methods of this disclosure include treating a subject with an NK-1R antagonist wherein the subject is in need of treatment, in order to provide the subject with a beneficial effect or improvement. In one embodiment, a method of this disclosure includes providing a subject with protection against a herpesviridae (HV) infection or pathogenesis by administering a composition comprising a sufficient amount of an NK-1R antagonist to provide the subject with protection against a herpesviridae (HV) infection or pathogenesis. In a further embodiment, these methods include treating a subject for herpesviridae (HV) infection or pathogenesis by administering a composition comprising a sufficient amount of an NK-1R antagonist to treat the subject for the herpesviridae (HV) infection or pathogenesis. These methods may include decreasing susceptibility of a subject to a herpesviridae (HV) infection or pathogenesis by administering a composition comprising a sufficient amount of an NK-1R antagonist to decrease susceptibility of the subject to a herpesviridae (HV) infection or pathogenesis. Methods of this disclosure include administering an NK-1R antagonist prior to, concurrently with, or following contact of the subject with, exposure of the subject to, infection with or reactivation of a herpesviridae (HV); and administering an NK-1R antagonist prior to, concurrently with, or following development of a symptom or pathology associated with or caused by herpesviridae (HV) infection or reactivation.

In various aspects, an NK-1R antagonist is administered prior to (prophylaxis), concurrently with, or following infection, contact or exposure of the subject to HV, or reactivation of HV (therapeutic).

These treatment methods therefore include, among other things, therapeutic and prophylactic methods. Subjects can be contacted with, administered ex vivo or in vivo, an NK-1R antagonist prior to, concurrently with, or following HV exposure or contact, HV infection, development of a symptom or pathology associated with or caused by a HV infection or pathogenesis, or reactivation of HV from latency.

The term “therapeutic,” and grammatical variations thereof, means the subject has a herpesviridae (HV) infection, for example, the subject exhibits one or more symptoms or pathologies associated with or caused by an acute or chronic HV infection, reactivation or pathogenesis as set forth herein or known in the art. The term “therapeutic” also includes a subject that has been exposed to or contacted with HV but may not exhibit one or more symptoms or pathologies associated with or caused by acute or chronic HV infection, reactivation or pathogenesis, as set forth herein or known in the art.

“Prophylaxis” and grammatical variations thereof refer to contact, administration, or in vivo delivery to a subject prior to a known contact with or exposure to herpesviridae (HV). In situations where it is not known if a subject has been contacted with or exposed to HV, contact with, administration or in vivo delivery of a compound to a subject occurs prior to manifestation or onset of a symptom associated with or caused by HV infection or pathogenesis. In such a method, the effect of contact with, administration or in vivo delivery of an NK-1R antagonist can be to eliminate, prevent, inhibit, decrease or reduce the probability of or susceptibility towards developing an HV infection, reactivation or pathogenesis, or a symptom or pathology associated with or caused by HV infection, reactivation or pathogenesis.

As used herein, the term “associated with,” when used in reference to the relationship between a symptom, pathology, or adverse side effect of herpesviridae (HV), means that the symptom, pathology or side effect is caused by HV infection, reactivation from latency, or pathogenesis, or is a secondary effect of HV infection, reactivation from latency, or pathogenesis. A symptom, pathology or side effect that is present in a subject may therefore be the direct result of or caused by the herpesviridae (HV) infection, reactivation or pathogenesis, or may be due at least in part to the subject reacting or responding to HV infection, reactivation, or pathogenesis (e.g., the immunological response). For example, a symptom or pathology that occurs during a herpesviridae (HV) infection, reactivation or pathogenesis may be due in part to an inflammatory response of the subject.

This disclosure also provides methods for decreasing or preventing an adverse side effect caused by vaccination of a subject with or against a herpesviridae (HV). In one embodiment, a method includes administering a sufficient amount of an NK-1R antagonist to the subject to decrease or prevent an adverse side effect caused by vaccination with a herpesviridae (HV). In one aspect, the herpesviridae (HV) comprises an alpha-, beta- or gamma-herpesvirus (e.g., herpes simplex virus-1 (HSV-1), herpes simplex virus-2 (HSV-2), varicella zoster virus (VZV/HHV-3).

Herpesviridae (HV) is typically found in biological fluids, cells, tissues or organs, in vivo. Accordingly, HV present in any biological fluid, cell, tissue or organ, is treatable with the NK-1R antagonist and methods, locally, regionally or systemically. For example, HV is present in a biological fluid (e.g., mucus, saliva, blood, serum, plasma, cerebrospinal fluid, urine, or placenta); in a tissue or organ comprising a transplant; in an immune cell, tissue or organ, mucosal cell, tissue or organ, neural cell, tissue or organ, or epithelial cell, tissue or organ. An immune cell may be a T cell or a B cell; a mucosal cell or tissue may be mouth, buccal cavity, labia, nasopharynx, esophagus, trachea, lung, stomach, small intestine, vagina, rectum, or colon; a neural cell or tissue may be ganglia, motor or sensory neuron; and an epithelial cell or tissue may be nose, fingers, ears, cornea, conjunctiva, skin or dermis.

An NK-1R antagonist useful in these methods may include one or more of aprepitant, rolapitant, fosaprepitant, lanepitant, befetupitant, or combinations thereof.

Methods of treatment include reducing, decreasing, inhibiting, ameliorating or preventing onset, severity, duration, progression, frequency, or probability of one or more adverse side effects associated with herpesviridae (HV) vaccination (e.g., a live or attenuated pathogenic or non-pathogenic HV, a vaccine comprising an HV protein, such as glycoprotein D, etc.). Non-limiting examples of adverse side effects associated with HV vaccination treatable with an NK-1R antagonist include fatigue, weakness, headache, fever, stomach ache/nausea, flu-like symptoms, rash, vomiting, inflammation (cerebral or ocular), and fainting.

Methods of this disclosure, including, for example, prophylactic and therapeutic treatment methods, as well as methods for decreasing or preventing an adverse side effect caused by vaccination with or against herpesvirus, are applicable to HV generally, more specifically, the members of the family Herpesviridae. Herpesviridae (HV) includes any strain or isolate or subtype or a species of HV, or combination of strains or isolates or subtypes or species of herpesviruses. Particular examples are infectious or pathogenic viruses. Specific non-limiting examples of HV include, for example, live or attenuated pathogenic and non-pathogenic HV. Exemplary HV include, alpha-, beta-, and gamma-herpesvirus. Particular non-limiting examples of alpha-virus include herpes simplex virus-1 (HSV-1), herpes simplex virus-2 (HSV-2) and varicella zoster virus (VZV/HHV-3).

Methods of this disclosure include methods of treatment that result in a beneficial effect in the subject receiving the treatment. Non-limiting examples of beneficial effects include providing a subject with partial or complete protection against HV infection, reactivation or pathogenesis, or a symptom caused by a HV infection, reactivation or pathogenesis (e.g., inhibit or reduce probability or susceptibility). Non-limiting examples of beneficial effects may also include reducing, decreasing, inhibiting, delaying, or preventing HV infection, reactivation or pathogenesis, and reducing, decreasing, inhibiting, ameliorating or preventing onset, severity, duration, progression, frequency or probability of one or more symptoms or pathologies associated with a HV infection, reactivation or pathogenesis. Additional non-limiting examples of beneficial effects also include reducing, decreasing amounts of, or inhibiting, delaying, or preventing increases in HV titer or viral load, proliferation or replication. Further non-limiting examples of beneficial effects include reducing, decreasing, inhibiting, delaying, ameliorating, or preventing onset, progression, severity, duration, frequency, probability, or susceptibility of a subject to HV infection, reactivation or pathogenesis, or accelerating, facilitating, or hastening recovery of a subject from HV infection, reactivation, or pathogenesis or one or more associated symptoms or pathologies.

Methods of this disclosure therefore include providing a beneficial or therapeutic effect to a subject, for example, reducing, decreasing, inhibiting, delaying, ameliorating or preventing onset, progression, severity, duration, frequency or probability of HV infection, reactivation or pathogenesis or one or more symptoms or pathologies associated with or caused by HV infection, reactivation or pathogenesis; reducing, decreasing, inhibiting, delaying or preventing increases in HV titer, viral load, replication, proliferation, or an amount of a viral protein of one or more HV strains or isolates or subtypes. Stabilizing the infection, reactivation, or a symptom or pathology thereof, or preventing, inhibiting or delaying reactivation, worsening or progression of infection, reactivation or a symptom or pathology associated with or caused by HV infection, reactivation or pathogenesis, or progression of the underlying HV infection, are also included in various embodiments of the methods of this disclosure.

Methods of this disclosure are applicable to providing a subject with protection against HV infection, reactivation, or pathogenesis, treating a subject for HV infection, reactivation and pathogenesis; and decreasing susceptibility or inhibiting HV reactivation from latency in a subject. The methods of this disclosure are therefore applicable to HV infection that is in an active state, latent state, or reactivated state.

The term “infection,” when used in reference to HV, means an initial or primary infection. An infection may be “infectious” in the sense that HV infects other sites in the infected host subject, or contagious to other subjects (cross-infection), or may be latent, in which case HV does not generally infect other sites or is contagious to other subjects. In immunocompetent subjects, initial/primary infection is usually either asymptomatic or causes mild pathogenesis or symptoms; only a small proportion of subjects develop more severe clinical illness. Primary infection is self-limiting in immunocompetent patients. In contrast, primary HV infection in immunocompromised subjects (e.g., immunosuppressant treatment, HIV+, newborns/neonates, pregnant, elderly subjects, etc.), can result in severe symptoms and may even be fatal.

Following a primary or initial HV infection, the virus establishes “latency,” in the host subject which allows the virus to evade immune clearance and remain in the host subject, and infection is lifelong. In the latent state, HV does not typically cause illness or symptoms, there is little if any viral replication and the subject is not infectious or contagious. Latency, also referred to as “latent infection” may occur in a different cell type from that of the initial/primary HV infection.

The term “reactivation,” when used in reference to HV, means activation of HV in the host subject following a period of latency. Reactivation is associated with increased viral replication and proliferation in an HV infected host subject, who becomes infectious and contagious again. Symptoms and pathologies associated with or caused by HV reactivation may or may not be the same type, severity, frequency, or duration as initial HV infection and subsequent pathogenesis. For example, VZV/HHV-3 causes chickenpox (primary infection) and shingles (reactivation). Reactivation can be milder (e.g., asymptomatic) than an initial HV infection/pathogenesis, in which case it would not be obvious whether a host subject is in a latent or reactivated state. In immunocompetent host subjects' reactivation is typically mild, whereas in immunocompromised host subjects, symptoms associated with or caused by reactivation can be severe and lead to death. Thus, clinical manifestations associated with reactivation may be different from that observed with an initial/primary infection. Accordingly, a single HV can cause different clinical symptoms or pathologies. One symptom of HV reactivation is the appearance of “cold sores” around mucosal areas (e.g., mouth, lips, tongue, genitalia, etc.). Reactivation occurs periodically and can be induced by stress, immune suppression, etc.

Specific examples of symptoms and pathologies associated with or caused by herpesviridae (HV) infection, reactivation or pathogenesis, whose onset, progression, severity, frequency, duration, or probability can be reduced, decreased, inhibited, delayed, ameliorated or prevented include, for example, lesions, ulcers, canker sore, cold sore, rash, boils, Gingivostomatitis, Herpetic whitlow Traumatic herpes (herpes gladiatorum), Eczema herpeticum, fever, fatigue, headache, sore throat, swollen lymph nodes, pneumonitis, pneumonia, hepatitis, meningitis, myelitis, myelopathy, neuropathy, pancreatitis, VZV-associated diabetic complications, vasculopathy, Encephalitis, keratitis, Genital herpes, esophagitis, dysphasia, hemiparesis, coma, shingles, chicken pox, mononucleosis, chronic or acute pelvic inflammatory disease (PID), proctitis, colitis, nerve damage and death. Other symptoms and pathologies of HV infection, reactivation or pathogenesis, are known in the art and treatment thereof in accordance with the methods of this disclosure is provided.

The methods of this disclosure, including, among other methods, providing a subject with protection against a herpesviridae (HV) infection, reactivation or pathogenesis, treatment of a herpesviridae (HV) infection, reactivation or pathogenesis, or a symptom or pathology associated with or caused by herpesviridae (HV) infection, reactivation or pathogenesis, or decreasing susceptibility of a subject to a herpesviridae (HV) infection, reactivation or pathogenesis, can therefore result in an improvement in the subjects' condition. An improvement is therefore any objective or subjective reduction, decrease, inhibition, delay, ameliorating, or prevention of onset, progression, severity, duration, frequency or probability of one or more symptoms or pathologies associated with or caused by HV infection, reactivation or pathogenesis (e.g., illness), or virus titer, viral load, replication, proliferation, or an amount of a viral protein. An improvement would also include reducing, inhibiting, or preventing increases in virus titer, viral load, replication, proliferation, or an amount of a viral protein of one or more HV strains or isolates or subtypes or species. An improvement would further include stabilizing a symptom or pathology associated with or caused by HV infection, reactivation, or pathogenesis, or inhibiting, decreasing, delaying, or preventing a worsening or progression of the symptom or pathology associated with or caused by HV infection, reactivation, or pathogenesis, or progression of the underlying HV infection. An improvement can therefore be, for example, in any of lesions, ulcers, canker sore, cold sore, rash, boils, Gingivostomatitis, Herpetic whitlow Traumatic herpes (herpes gladiatorum), Eczema herpeticum, fever, fatigue, headache, sore throat, swollen lymph nodes, pneumonitis, pneumonia, hepatitis, meningitis, myelitis, myelopathy, neuropathy, pancreatitis, VZV-associated diabetic complications, vasculopathy, Encephalitis, keratitis, Genital herpes, esophagitis, dysphasia, hemiparesis, coma, shingles, chicken pox, mononucleosis, chronic or acute pelvic inflammatory disease (PID), proctitis, colitis, nerve damage, and death to any degree or for any duration of time (hours, days, weeks, months, years, or cure).

An improvement would also include reducing or eliminating a need, dosage amount, or frequency of another treatment, such as an antiviral drug or other agent used for treating a subject having, or at risk of having, a herpesviridae (HV) infection, reactivation, or pathogenesis, a symptom or pathology associated with or caused by herpesviridae (HV) infection, reactivation or pathogenesis, or decreasing or preventing an adverse side effect caused by vaccination with or against a herpesviridae (HV). Thus, reducing an amount of another treatment for HV infection, reactivation or pathogenesis, a symptom or pathology associated with or caused by HV, or an adverse side effect caused by vaccination with or against a HV is considered to provide a benefit and, therefore, is considered within the methods of this disclosure. Non-limiting exemplary HV treatments that may be eliminated or used at reduced doses or frequencies of administration include protease inhibitors, reverse transcriptase inhibitors, virus fusion inhibitors, and virus entry inhibitors.

A treatment or improvement need not be complete ablation of any particular infection, reactivation, pathogenesis, symptom, pathology or adverse side effect, or all of the infection, reactivation, pathology, symptoms, pathologies or adverse side effects associated with or caused by HV infection, reactivation or pathogenesis, or vaccination with or against HV. Rather, treatment may be any objective or subjective measurable or detectable anti-virus effect or improvement in a treated subject. Thus, reducing, inhibiting, decreasing, eliminating, delaying, halting, or preventing a progression or worsening of the infection, reactivation or pathogenesis, a symptom or pathology of the infection, or an adverse side effect caused by vaccination is a satisfactory outcome. For example, an NK-1R antagonist may reduce, inhibit, delay formation of, or stabilize lesions, ulcers, canker sores, or cold sores, but not have a measurable effect on rash, boils, Gingivostomatitis, Herpetic whitlow Traumatic herpes (herpes gladiatorum), Eczema herpeticum, fever, fatigue, headache, sore throat, swollen lymph nodes, pneumonitis, pneumonia, hepatitis, meningitis, myelitis, myelopathy, neuropathy, pancreatitis, VZV-associated diabetic complications, vasculopathy, Encephalitis, keratitis, genital herpes, esophagitis, dysphasia, hemiparesis, coma, shingles, chicken pox, mononucleosis, chronic or acute pelvic inflammatory disease (PID), proctitis, colitis, nerve damage or death. Another example is where an NK-1R antagonist reduces fever or fatigue, without a detectable improvement in one or more other symptoms or pathologies. Thus, a satisfactory clinical endpoint is achieved when there is an incremental improvement in the subject's condition or a partial reduction or a stabilization of a HV infection, reactivation, pathogenesis or a symptom, pathology or adverse side effect thereof, or an inhibition or prevention of worsening or progression of the HV infection, reactivation, pathogenesis, symptom, pathology or adverse side effect thereof (stabilizing one or more symptoms or pathologies), over a short or long duration (hours, days, weeks, months, years, or cure).

In the methods of this disclosure in which there is a desired outcome, for example, a therapeutic or prophylactic method that provides an objective or subjective improvement in a HV infection, reactivation or pathogenesis, a symptom or pathology associated with or caused by HV, or an adverse side effect caused by vaccination with or against HV or an HV treatment, an NK-1R antagonist may be administered in a sufficient or effective amount. As used herein, a “sufficient amount” or “effective amount” or an “amount sufficient” or an “amount effective” refers to an amount that provides, in single or multiple doses, alone or in combination with one or more other compounds, treatments, agents (e.g., a drug) or therapeutic regimens, a long term or a short term detectable or measurable improvement or beneficial effect to a given subject of any degree or for any time period or duration (e.g., for minutes, hours, days, months, years, or cured). A “sufficient amount” or “effective amount” therefore includes an amount sufficient to result in decreasing, reducing, inhibiting, preventing, or delaying onset; decreasing, reducing, inhibiting, delaying, or preventing a progression or worsening of; or reducing, relieving, ameliorating, or alleviating, severity, frequency, duration, susceptibility, or probability of HV infection, reactivation or pathogenesis, one or more symptoms associated with or caused by HV infection, reactivation or pathogenesis, or an adverse side effect of vaccination with or against a HV or an HV treatment. In addition, hastening a subject's recovery from HV infection, reactivation or pathogenesis, one or more symptoms associated with or caused by HV infection, reactivation or pathogenesis, or an adverse side effect of vaccination with or against a HV or an HV treatment is considered to be a sufficient or effective amount. Various beneficial effects and indicia of therapeutic and prophylactic benefit are set forth herein and are known to the skilled artisan.

A sufficient amount or an effective amount can but need not be provided in a single administration and can but need not be administered alone (i.e., without a second drug, agent, treatment or therapeutic regimen), or in combination with another compound, agent, treatment or therapeutic regimen. In addition, a sufficient amount or an effective amount need not be sufficient or effective if given in single or multiple doses without a second compound, treatment, agent, or therapeutic regimen, because additional doses, amounts, frequency or duration of administration above and beyond such doses, or additional compounds, agents, treatments or therapeutic regimens may be included in order to be effective or sufficient in a given subject.

A sufficient amount or an effective amount need not be effective in each and every subject, nor a majority of subjects in a given group or population. Thus, a sufficient amount or an effective amount means sufficiency or effectiveness in a particular subject, not a group or the general population. As is typical for such methods, some subjects will exhibit a greater or lesser response to a method of this disclosure than other subjects.

Amounts, frequencies, or duration also considered sufficient and effective and are therefore beneficial are those that result in the elimination or a reduction in amount, frequency or duration of another compound, agent, treatment or therapeutic regimen. For example, an NK-1R antagonist is considered as having a beneficial or therapeutic effect if contact, administration or delivery in vivo results in the use of a lesser amount, frequency or duration of another compound, agent, treatment or therapeutic regimen to treat the infection, pathogenesis, symptom or pathology, or adverse side effect of vaccination.

Any compound, agent, treatment (e.g., a biologically active ingredient) or other therapeutic regimen having a beneficial, additive, synergistic or complementary activity or effect can be formulated or used in combination with or in addition to the NK-1R antagonist. In various embodiments, the compound, agent, treatment, or therapeutic regimen is for providing a subject with protection against HV infection, reactivation or pathogenesis; decreasing susceptibility of a subject to a HV infection, reactivation or pathogenesis; or decreasing or preventing an adverse side effect caused by HV vaccination or an HV treatment.

Examples of such combination compositions and methods include protease inhibitors, reverse transcriptase inhibitors, virus fusion inhibitors and virus entry inhibitors.

The term “subject” refers to an animal, typically mammalian animals, such as but not limited to non-human primates (apes, gibbons, gorillas, chimpanzees, orangutans, macaques), domestic animals (dogs and cats), a farm animals (chickens, ducks, horses, cows, goats, sheep, pigs), experimental animal (mouse, rat, rabbit, guinea pig) and humans. Subjects include animal models, for example, a mouse model of herpesvirus infection (e.g., alpha, beta-, or gamma-herpesvirus). Subjects include naturally occurring or non-naturally occurring mutated or non-human genetically engineered (e.g., transgenic or knockout) animals. Subjects further include animals having or at risk of having a chronic or acute HV infection, reactivation or pathogenesis, symptom or pathology of HV infection, reactivation or pathogenesis, or adverse side effect caused by vaccination with or against HV or an HV treatment. Subjects can be any age. For example, a subject (e.g., human) can be a newborn, infant, toddler, child, teenager, or adult, e.g., 50 years or older.

Subjects include those in need of therapeutic methods of this disclosure, e.g., in need of a therapeutic or prophylactic treatment. A subject is considered to be in need of a therapeutic method of this disclosure where a method is likely to provide some benefit to a subject. Various benefits provided to a subject are as set forth herein and known in the art for HV infection, reactivation or pathogenesis, symptoms, or pathologies caused by or associated with HV infection, reactivation or pathogenesis, and adverse side effects caused by vaccination with or against a HV or treatment of HV. Subjects appropriate for treatment include those having HV infection, reactivation or pathogenesis or currently or previously having any symptom or pathology associated with or caused by HV infection, reactivation or pathogenesis (e.g., diagnosed as HV+), HV vaccination or an HV treatment. Target subjects therefore include subjects infected with HV that are infectious or contagious, subjects infected with HV that is in a latent state, and subjects in which HV is or has been reactivated from latency. Thus, subjects that have been exposed to a HV (e.g., subjects that do produce an antibody against an HV protein) are appropriate targets. Such subjects may or may not have developed one or more adverse symptoms or pathologies associated with, or caused by, HV infection, reactivation or pathogenesis, regardless of the virus type, timing or degree of onset, progression, severity, frequency, duration of any infection, pathogenesis, symptom, pathology or adverse side effect. A subject may therefore be symptomatic or asymptomatic for HV infection, reactivation or pathogenesis.

Subjects appropriate for treatment also include those at risk of HV infection, reactivation or pathogenesis or at risk of having or developing a symptom or pathology associated with, or caused by, HV infection, reactivation, or pathogenesis. Candidate subjects therefore include subjects that have been exposed to or contacted with HV, or that are at risk of exposure to or contact with HV, regardless of the type, timing, or extent of exposure or contact. These methods are therefore applicable to a subject who is at risk of HV infection, reactivation or pathogenesis, but has not yet been exposed to, or contacted with, herpesviridae (HV). Thus, subjects that have not been exposed to HV (e.g., subjects that do not produce an antibody against an HV protein) are appropriate targets. Prophylactic methods are therefore included. Subjects targeted for prophylaxis can be at increased risk (probability or susceptibility) of herpesviridae (HV) infection or pathogenesis, as set forth herein and known in the art.

At risk subjects appropriate for treatment include subjects exposed to other subjects having an HV infection or reactivation (infectious or contagious), or where the risk of HV infection is increased due to changes in virus infectivity or cell tropism, immunological susceptibility (e.g., an immunocompromised subject), or environmental risk. At risk subjects appropriate for treatment therefore include human subjects exposed to, or at risk of, exposure to other humans that have HV infection or reactivation (infectious or contagious), or are at risk of a HV infection or reactivation (infectious or contagious).

Subjects further include immunocompromised subjects due to an immunological disorder (e.g., autoimmunity) or disease, or an immune-suppressing treatment (e.g., cyclophosphamide). Subjects also include those having been exposed to or diagnosed as HIV+. Subjects further include those receiving or candidates for a tissue or organ transplant.

The NK-1R antagonist compounds useful in the methods of this disclosure can be incorporated into pharmaceutical compositions or formulations. Such pharmaceutical compositions/formulations are useful for administration to a subject, in vivo or ex vivo. Pharmaceutical compositions and formulations include carriers or excipients for administration to a subject. As used herein the terms “pharmaceutically acceptable” and “physiologically acceptable” mean a biologically compatible formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically-acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions. The formulations may, for convenience, be prepared or provided as a unit dosage form. In general, formulations are prepared by uniformly and intimately associating the active ingredient with liquid carriers or finely divided solid carriers or both, and then, if necessary, shaping the product. For example, a tablet may be made by compression or molding. Compressed tablets may be prepared by compressing, in a suitable machine, an active ingredient in a free-flowing form such as a powder or granules, optionally mixed with a binder, lubricant, inert diluent, preservative, surface-active or dispersing agent. Molded tablets may be produced by molding, in a suitable apparatus, a mixture of powdered compound moistened with an inert liquid diluent. The tablets may optionally be coated or scored and may be formulated to provide a slow or controlled release of the active ingredient therein.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethylene glycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone. Supplementary active compounds (e.g., preservatives, antioxidants, antiviral or antimicrobial agents including biocides and biostats such as antibacterial, antiviral, and antifungal agents) can also be incorporated into the compositions. Preservatives and other additives include, for example, antimicrobials, anti-oxidants, chelating agents and inert gases (e.g., nitrogen). Pharmaceutical compositions may therefore include preservatives, antimicrobial agents, anti-oxidants, chelating agents, and inert gases.

Preservatives can be used to inhibit microbial growth or increase stability of the active ingredient thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

Pharmaceutical compositions can optionally be formulated to be compatible with a particular route of administration. Exemplary routes of administration include administration to a biological fluid, an immune cell (e.g., T or B cell) or tissue, mucosal cell or tissue (e.g., mouth, buccal cavity, labia, nasopharynx, esophagus, trachea, lung, stomach, small intestine, vagina, rectum, or colon), neural cell or tissue (e.g., ganglia, motor or sensory neurons) or epithelial cell or tissue (e.g., nose, fingers, ears, cornea, conjunctiva, skin or dermis). Thus, pharmaceutical compositions include carriers (excipients, diluents, vehicles or filling agents) suitable for administration to any cell, tissue or organ, in vivo, ex vivo (e.g., tissue or organ transplant) or in vitro, by various routes and delivery locally, regionally, or systemically.

Exemplary routes of administration for contact or in vivo delivery that an NK-1R antagonist can optionally be formulated for include inhalation, respiration, intubation, intrapulmonary instillation, oral (buccal, sublingual, mucosal), intrapulmonary, rectal, vaginal, intrauterine, intradermal, topical, dermal, parenteral (e.g., subcutaneous, intramuscular, intravenous, intradermal, intraocular, intratracheal and epidural), intranasal, intrathecal, intraarticular, intracavity, transdermal, iontophoretic, ophthalmic, optical (e.g., corneal), intraglandular, intraorgan, intralymphatic.

Formulations suitable for parenteral administration include aqueous and non-aqueous solutions, suspensions or emulsions of the compound, which may include suspending agents and thickening agents, which preparations are typically sterile and can be isotonic with the blood of the intended recipient. Non-limiting illustrative examples of aqueous carriers include water, saline (sodium chloride solution), dextrose (e.g., Ringer's dextrose), lactated Ringer's, fructose, ethanol, animal, vegetable or synthetic oils. Examples of non-aqueous solvents are propylene glycol, polyethylene glycol, vegetable oils such as olive oil, and injectable organic esters such as ethyl oleate. Intravenous vehicles include fluid and nutrient replenishers, electrolyte replenishers (such as those based on Ringer's dextrose). The formulations may be presented in unit-dose or multi-dose kits, for example, ampules and vials, and may be stored in a freeze-dried (lyophilized) condition requiring addition of a sterile liquid carrier, for example, water for injection, prior to use.

For transmucosal or transdermal administration (e.g., topical contact), penetrants can be included in the pharmaceutical composition. Penetrants are known in the art and include, for example, for transmucosal administration, detergents, bile salts, and fusidic acid derivatives. For transdermal administration, the active ingredient can be formulated into aerosols, sprays, ointments, salves, gels, pastes, lotions, oils, or creams as generally known in the art.

For topical administration, for example, to skin, pharmaceutical compositions typically include ointments, creams, lotions, pastes, gels, sprays, aerosols or oils. Carriers which may be used include white petrolatum, lanolin, polyethylene glycols, alcohols, transdermal enhancers, and combinations thereof. An exemplary topical delivery system is a transdermal patch containing an active ingredient.

For oral administration, pharmaceutical compositions include capsules, cachets, lozenges, tablets or troches, as powder or granules. Oral administration formulations also include a solution or a suspension (e.g., aqueous liquid or a non-aqueous liquid; or as an oil-in- water liquid emulsion or a water-in-oil emulsion).

For airway or nasal administration, pharmaceutical compositions can be formulated in a dry powder for delivery, such as a fine or a coarse powder having a particle size, for example, in the range of 20 to 500 microns which is administered in the manner by inhalation through the airways or nasal passage. Depending on delivery device efficiency, effective dry powder dosage levels typically fall in the range of about 10 to about 100 mg. Appropriate formulations, wherein the carrier is a liquid, for administration, as for example, a nasal spray or as nasal drops, include aqueous or oily solutions of the active ingredient.

For airway or nasal administration, aerosol and spray delivery systems and devices, also referred to as “aerosol generators” and “spray generators,” such as metered dose inhalers (MDI), nebulizers (ultrasonic, electronic and other nebulizers), nasal sprayers and dry powder inhalers can be used. MDIs typically include an actuator, a metering valve, and a container that holds a suspension or solution, propellant, and surfactant (e.g., oleic acid, sorbitan trioleate, lecithin). Activation of the actuator causes a predetermined amount to be dispensed from the container in the form of an aerosol, which is inhaled by the subject. MDIs typically use liquid propellant and typically, MDIs create droplets that are 15 to 30 microns in diameter, optimized to deliver doses of 1 microgram to 10 mg of a therapeutic. Nebulizers are devices that turn medication into a fine mist inhalable by a subject through a face mask that covers the mouth and nose. Nebulizers provide small droplets and high mass output for delivery to upper and lower respiratory airways. Typically, nebulizers create droplets down to about 1 micron in diameter.

Dry-powder inhalers (DPI) can be used to deliver the NK-1R receptor antagonists, either alone or in combination with a pharmaceutically acceptable carrier. DPIs deliver active ingredient to airways and lungs while the subject inhales through the device. DPIs typically do not contain propellants or other ingredients, only medication, but may optionally include other components. DPIs are typically breath-activated, but may involve air or gas pressure to assist delivery.

For rectal administration, pharmaceutical compositions can be included as a suppository with a suitable base comprising, for example, cocoa butter or a salicylate. For vaginal administration, pharmaceutical compositions can be included as pessaries, tampons, creams, gels, pastes, foams or spray formulations containing in addition to the active ingredient a carrier, examples of appropriate carriers which are known in the art.

Pharmaceutical formulations and delivery systems appropriate for the compositions and methods of this disclosure are known in the art (see, e.g., Remington: The Science and Practice of Pharmacy (2003) 20.sup.th ed., Mack Publishing Co., Easton, Pa.; Remington's Pharmaceutical Sciences (1990) 18.sup.th ed., Mack Publishing Co., Easton, Pa.; The Merck Index (1996) 12.sup.th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11.sup.th ed., Lippincott Williams & Wilkins, Baltimore, Md.; and Poznansky et al., Drug Delivery Systems (1980), R. L. Juliano, ed., Oxford, N.Y., pp. 253-315).

The NK-1R antagonists may be packaged in unit dosage forms for ease of administration and uniformity of dosage. A “unit dosage form” as used herein refers to a physically discrete unit suited as unitary dosages for the subject to be treated; each unit containing a predetermined quantity of compound optionally in association with a pharmaceutical carrier (excipient, diluent, vehicle or filling agent) which, when administered in one or more doses, is calculated to produce a desired effect (e.g., prophylactic or therapeutic effect or benefit). Unit dosage forms can contain a daily dose or unit, daily sub-dose, or an appropriate fraction thereof, of an administered compound. Unit dosage forms also include, for example, capsules, troches, cachets, lozenges, tablets, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Unit dosage forms further include compounds for transdermal administration, such as “patches” that contact the epidermis of the subject for an extended or brief period of time. The individual unit dosage forms can be included in multi-dose kits or containers. Pharmaceutical formulations can be packaged in single or multiple unit dosage forms for ease of administration and uniformity of dosage.

In the methods of this disclosure, the NK-1R antagonist(s) may be administered in accordance with the methods at any frequency as a single bolus or multiple dose e.g., one, two, three, four, five, or more times hourly, daily, weekly, monthly or annually or between about 1 to 10 days, weeks, months, or for as long as appropriate. Exemplary frequencies are typically from 1-7 times, 1-5 times, 1-3 times, 2-times or once, daily, weekly or monthly. Timing of contact, administration ex vivo, or in vivo delivery can be dictated by the infection, reactivation, pathogenesis, symptom, pathology, or adverse side effect to be treated. For example, an amount can be administered to the subject substantially contemporaneously with, or within about 1-60 minutes or hours of the onset of a symptom or adverse side effect of HV infection, reactivation, pathogenesis, vaccination, or treatment.

Doses may vary depending upon whether the treatment is therapeutic or prophylactic, the onset, progression, severity, frequency, duration, probability of, or susceptibility of the symptom, the type of virus infection, reactivation or pathogenesis to which treatment is directed, clinical endpoint desired, previous, simultaneous or subsequent treatments, general health, age, gender or race of the subject, bioavailability, potential adverse systemic, regional or local side effects, the presence of other disorders or diseases in the subject, and other factors that will be appreciated by the skilled artisan (e.g., medical or familial history). Dose amount, frequency, or duration may be increased or reduced, as indicated by the clinical outcome desired, status of the infection, reactivation, pathology or symptom, or any adverse side effects of the treatment or therapy. The skilled artisan will appreciate the factors that may influence the dosage, frequency and timing required to provide an amount sufficient or effective for providing a prophylactic or therapeutic effect or benefit.

Typically, for therapeutic treatment, the NK-1R antagonist(s) will be administered as soon as practical, typically within 0-72 hours after a subject is exposed to, or contacted with, HV, or within 0-72 hours after development of one or more symptoms or pathologies associated with HV infection, reactivation, or pathogenesis (e.g., onset of lesions, ulcers, canker sores, cold sores, rash, boils, etc.) or a symptom associated with, or caused by, HV. For prophylactic treatment, an NK-1R antagonist may be administered immediately or within 0-72 after suspected contact with, or 0-4 weeks, e.g., 1-3 weeks, prior to anticipated or possible exposure to, or contact or infection with, or reactivation of, HV. For prophylactic treatment in connection with immunization or vaccination of a subject, an NK-1R antagonist can be administered prior to, concurrently with or following immunization/vaccination of the subject.

Doses can be based upon current existing treatment protocols (e.g., acyclovir), empirically determined, determined using animal disease models, or optionally in human clinical studies. The NK-1R antagonist may be administered to a subject in single bolus or in divided/metered doses, which can be adjusted to be more or less according to the various consideration set forth herein and known in the art. Dose amount, frequency or duration may be increased or reduced, as indicated by the status of the HV infection, reactivation or pathogenesis, associated symptom or pathology, or any adverse side effect(s) of vaccination, treatment or anti-HV therapy. For example, once control or a particular endpoint is achieved, (for example, reducing, decreasing, inhibiting, ameliorating, or preventing onset, severity, duration, progression, frequency, or probability of one or more symptoms associated with a HV infection, reactivation or pathogenesis of one or more symptoms or pathologies associated with or caused by HV infection, reactivation or pathogenesis), dose amount, frequency, or duration may be reduced. Each publication or patent cited herein is incorporated herein by reference in its entirety.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

The following methods and materials were used in conducting the experimental Examples described below.

Cell Culture and Infection: Primary human spinal astrocytes (HA-sp; Sciencell, Carlsbad, Calif.) were seeded at 5,000 cells/cm' in a basal astrocyte medium containing 2% fetal bovine serum (FBS), 1% astrocyte growth supplement, and 1% 100× penicillin-streptomycin (Sciencell). After 24 hours, medium was changed to basal astrocyte medium containing 0.1% FBS and 1% 100× penicillin-streptomycin that was replenished every 72 hours for 7 days to establish quiescence. On day 7, spinal astrocytes were co-cultivated with VZV-infected spinal astrocytes (40 pfu/mL) or mock-infected spinal astrocytes.
Aprepitant Application: HA-sp were VZV-infected as described above and incubated for 12 hours to establish productive infection. At 12 hours post-infection (HPI), VZV-infected spinal astrocytes were treated with 10 μM aprepitant (the optimal concentration to avoid cell death was determined in a preliminary toxicity assay on uninfected HA-sp using aprepitant at 1-100 μM; data not shown) or vehicle (DMSO) and treated again at 24 HPI and 48 HPI. Light microscopy images to observe a cytopathic effect were obtained at 12, 24, 48 and 72 HPI followed by DNA extraction and PCR quantification of VZV DNA as described below.
Substance P ELISA: Substance P was quantitated from mock- and VZV-infected HA-sp using a colorimetric competitive enzyme immunoassay kit according to the manufacturer's instructions (Enzo, Farmingdale, N.Y.). The sensitivity range was 9.76-10,000 pg/mL, as determined by controls run on the same assay as samples.
Immunofluorescence Assay: HA-sp were propagated and treated as described above in an ibidi 24-well μ-Plate (ibidi, Martinsried, Germany). Spinal astrocytes were fixed with 4% paraformaldehyde for 20 minutes at room temperature and blocked with normal donkey serum (10%) for 1 hour. Cells were stained with a 1:500 dilution of mouse anti-human VZV-glycoprotein E (gE, Santa Cruz Biotechnology, Santa Cruz, Calif.) or a 1:500 dilution of mouse anti-human VZV-glycoprotein B (gB, Abcam, Cambridge, Mass.) for detection of specific VZV glycoproteins. GFAP and NK-1R were detected using a 1:500 dilution of chicken anti-GFAP (Abcam) and a 1:100 dilution of rabbit anti-NK-1R (Novus Biologicals LLC, Littleton, Colo.), respectively. Secondary antibodies consisted of Alexa Fluor 488 donkey anti-rabbit IgG, Alexa Fluor 594 donkey anti-mouse IgG, and Alexa Fluor 647 donkey anti-chicken IgG (MilliporeSigma, Burlington, Mass.), all at a 1:500 dilution. Following secondary antibody application and phosphate-buffered saline (PBS) washes, DAPI (4′,6-diamidino-2-phenylindole) (Vector Laboratories, Burlingame, Calif.) was added at a 1:500 dilution for 5 minutes, washed in PBS, then ibidi chambers visualized by microscopy. As a control for normal NK-1R localization when bound to its endogenous ligand, substance P, quiescent uninfected spinal astrocytes were treated with 10⁻⁶M substance P (Abcam, Cambridge, Mass.) for 60 minutes then visualized for process formation with CellTracker Deep Red (Invitrogen; Carlsbad, Calif.) or fixed and examined for the distribution of NK-1R by immunofluorescence.
DNA Extraction and Quantification: Mock- and VZV-infected HA-sp were harvested and placed into 200 μL of lysis buffer with proteinase K and incubated for 20 minutes at 56° C. (DNeasy Blood and Tissue Kit; Qiagen, Germantown, Md.). DNA was extracted per the manufacturer's instructions and eluted in 100 μL of nuclease-free water. DNA was then analyzed by quantitative PCR using primers corresponding to sequences in VZV ORF 68 and in cellular glyceraldehyde-3-phosphate-dehydrogenase (GAPdH) as previously described (Cohrs and Gilden, J Virol 2007; 81: 2950-6). Data were normalized to GAPDH and analyzed using the delta delta threshold cycle (C_{T}) method.
Statistical Analysis: Statistical analysis was performed using GraphPad Prism (GraphPad, San Diego, Calif.). Significance of differences in VZV DNA between treated and untreated VZV-infected HA-sp at each time point was determined using the Student's paired t test.

### Example 1

### Primary Human Spinal Astrocytes (HA-sp) are Permissive to VZV Infection

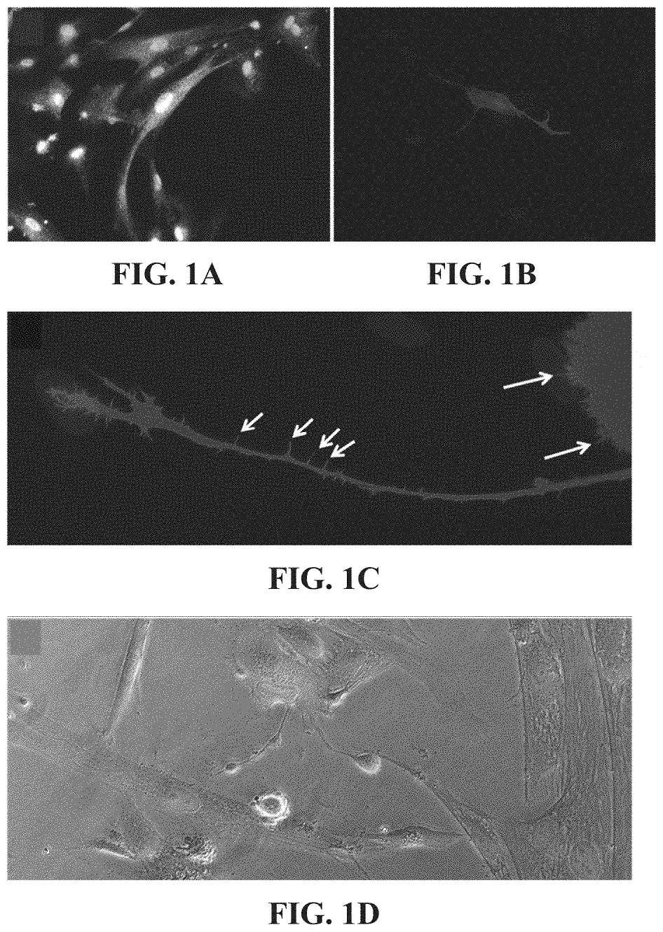

At 3 days post-infection (DPI), immunofluorescence analysis of mock-infected HA-sp using an antibody directed against glial fibrillary protein (GFAP, an astrocyte marker) revealed GFAP expression in all cells, indicating a pure astrocyte culture (FIG. 1A). Analysis of VZV-infected cells using an antibody directed against VZV gB indicated that spinal astrocytes were permissive to VZV infection, as shown by the infected spinal astrocyte with elongated cell processes (lamellipodia) contacting adjacent uninfected cells (FIG. 1B). Higher magnification of a VZV-infected spinal astrocyte (FIG. 1C) showed the striking morphology of the lamellipodia, which contained filopodia (short arrows) that also sprouted from the cell body (long arrows). Note the abundance of VZV gB along the lamellipodia and filopodia. A phase-contrast image showed the normal morphology of uninfected spinal astrocytes and altered morphology of a VZV-infected spinal astrocyte (FIG. 1C).

### Example 2

### VZV Infection is Associated with Neurokinin-1 Receptor Localization to the Nucleus in the Absence of the Endogenous Ligand, Substance P

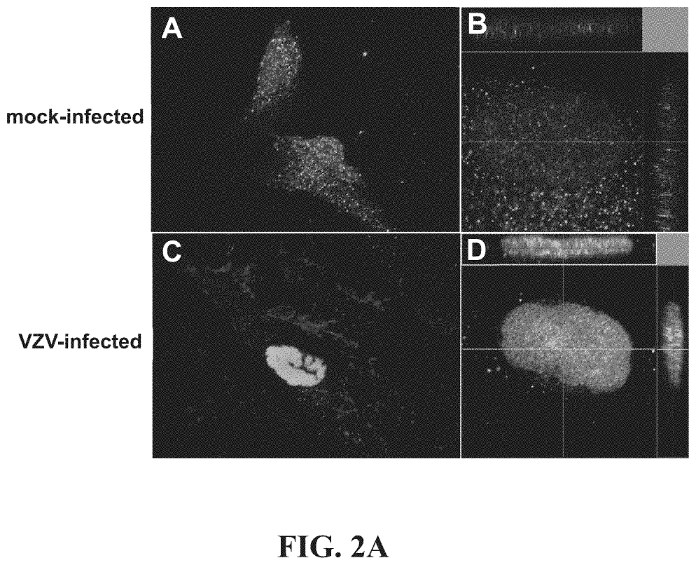

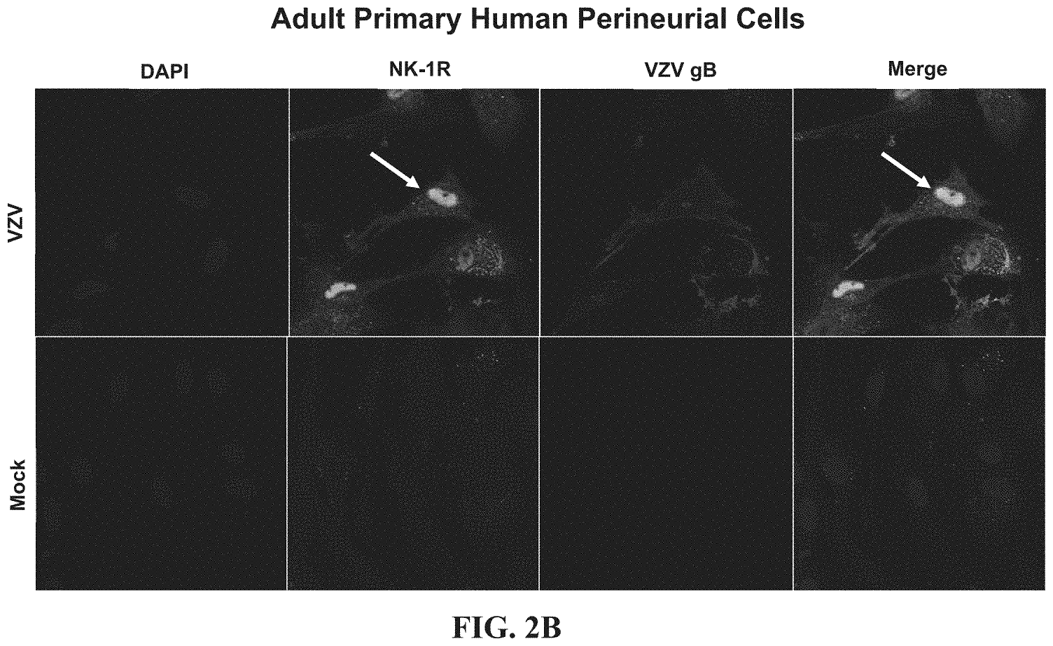

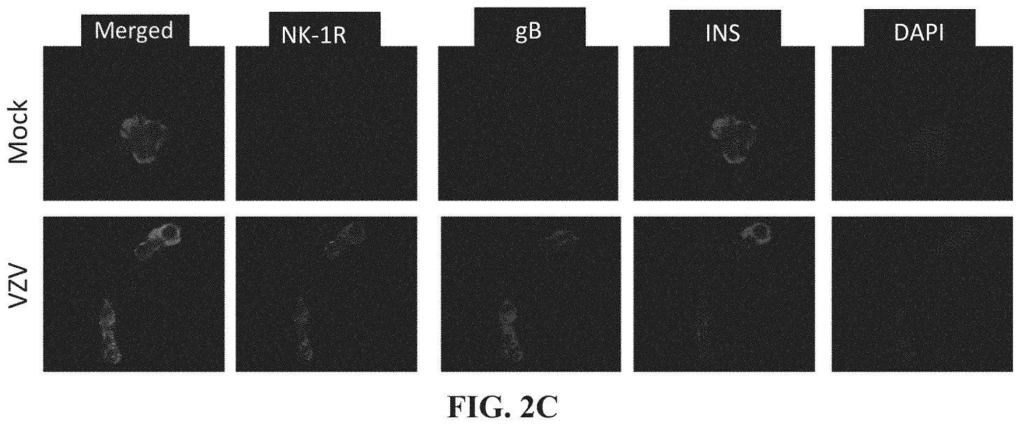

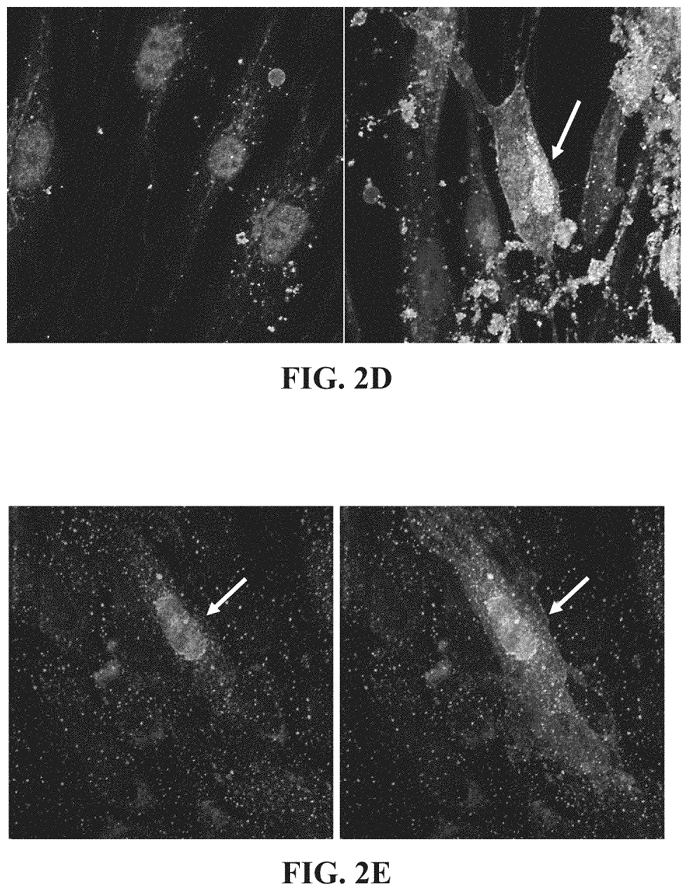

At 3 DPI, immunocytochemical analysis of mock-infected HA-sp using antibodies directed against NK-1R and VZV gE revealed NK-1R diffusely distributed in the cytoplasm and no VZV gE (FIG. 2, panels A and B), whereas VZV-infected cells expressed NK-1R predominantly in the nucleus and VZV gE diffusely (FIG. 2, panels C and D; nuclear z-stack image). Analysis of supernatant by ELISA revealed no substance P in mock- or VZV-infected cultures (values below lower limit of assay detection, <9.76 pg/mL).

Similarly, as shown in FIG. 2B, Neurokinin-1 receptor goes nuclear (arrows in top panels, columns 2 and 4) in VZV-infected adult primary human perineurial cells (HPNCs), facilitating viral spread.

### Example 3

### Substance P Does Not Induce NK-1R Nuclear Localization or Lamellipodia Formation

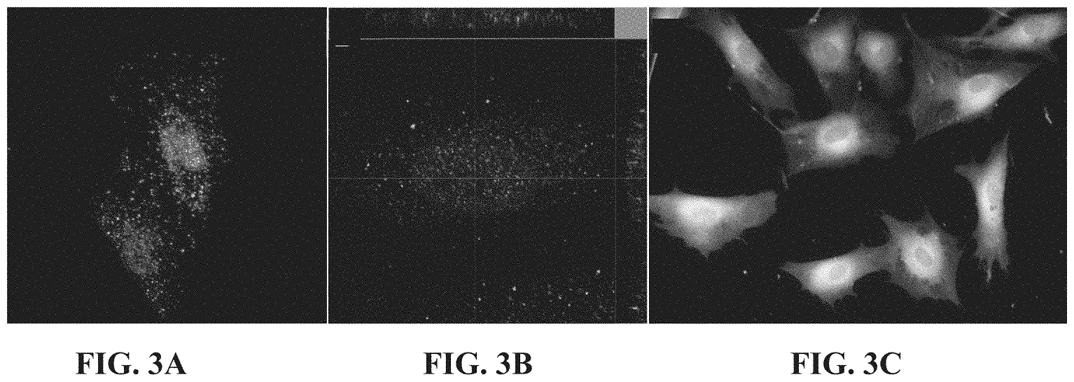

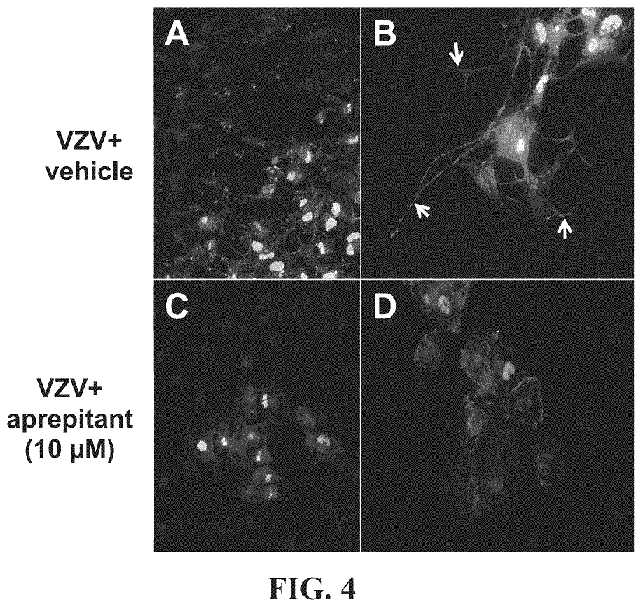

In uninfected spinal astrocytes treated with substance P, NK-1R was detected predominantly in cytoplasm (FIGS. 3A and 3B, nuclear z-stack image), unlike the presence of NK-1R in nucleus of VZV-infected cells (FIG. 2, panels C and D). The lack of lamellipodia in substance P-treated spinal astrocytes is shown by staining with CellTracker Deep Red (FIG. 3C).

### Example 4

### Aprepitant, an NK-1R Antagonist, Blocks Formation of Processes in VZV-Infected Spinal Astrocytes

At 3 DPI, immunocytochemical analysis of VZV-infected cells treated with vehicle revealed long, extended lamellipodia processes and predominantly nuclear NK-1R (FIG. 4, panels A and B); uninfected bystander cells did not have NK-1R in the nucleus or lamellipodia. In contrast, VZV-infected cells treated with vehicle plus 10 μM aprepitant showed reduction in lamellipodia formation, but retention of nuclear NK-1R in some cells (FIG. 4, panels C and D).

### Example 5

### Aprepitant Treatment after VZV Infection Reduces Viral DNA

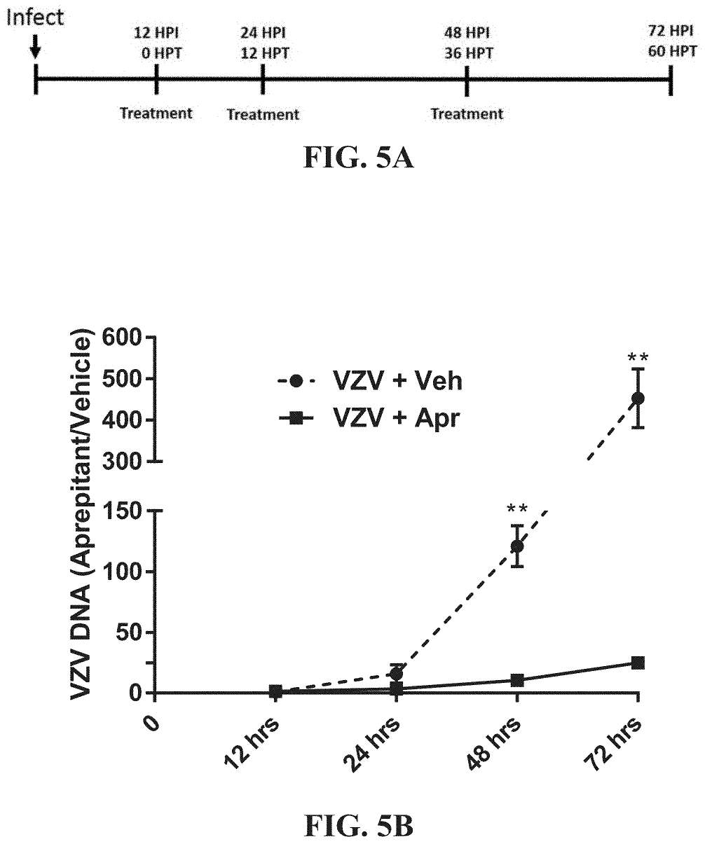

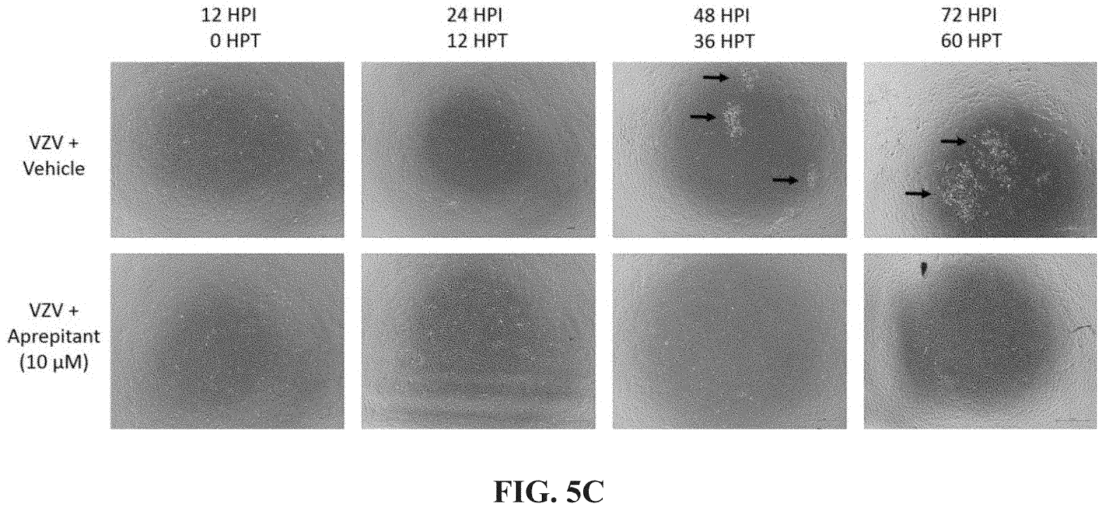

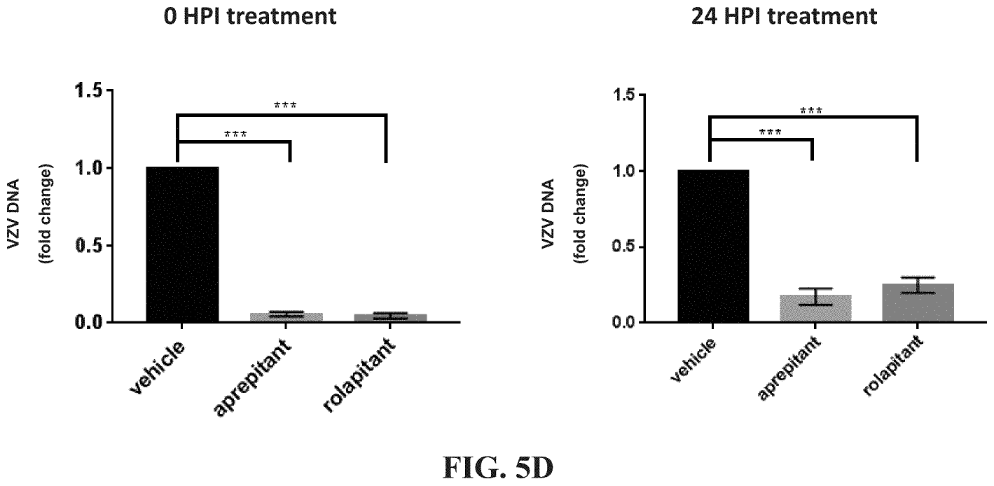

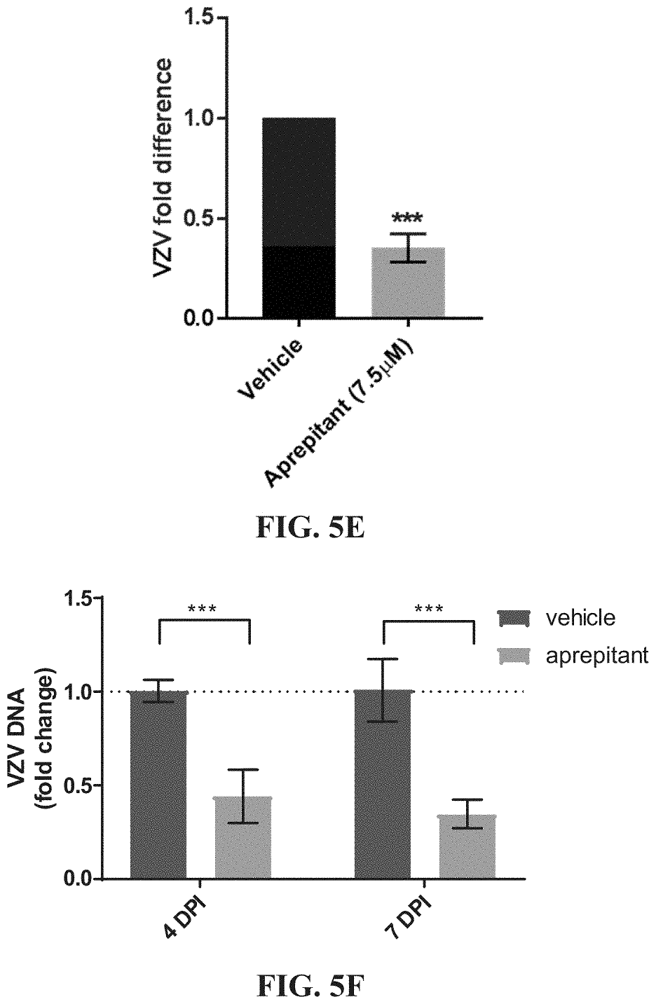

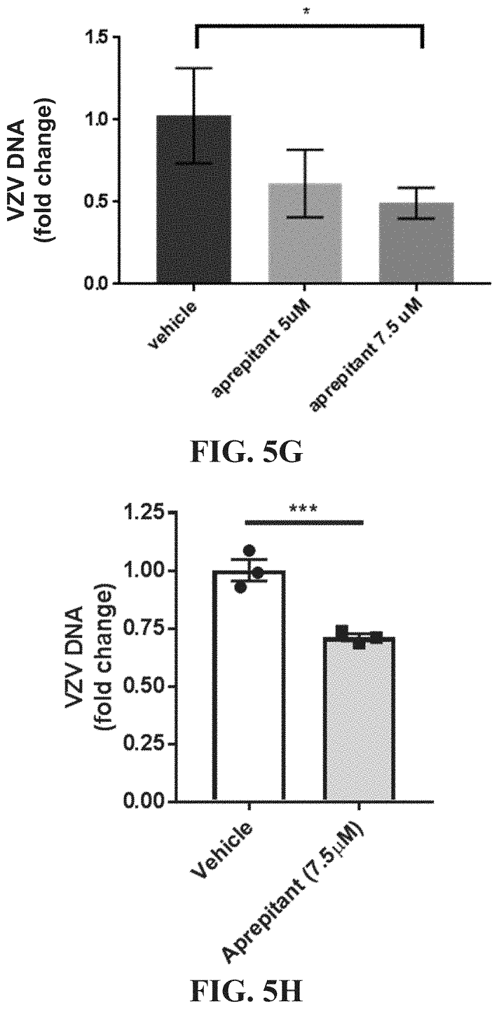

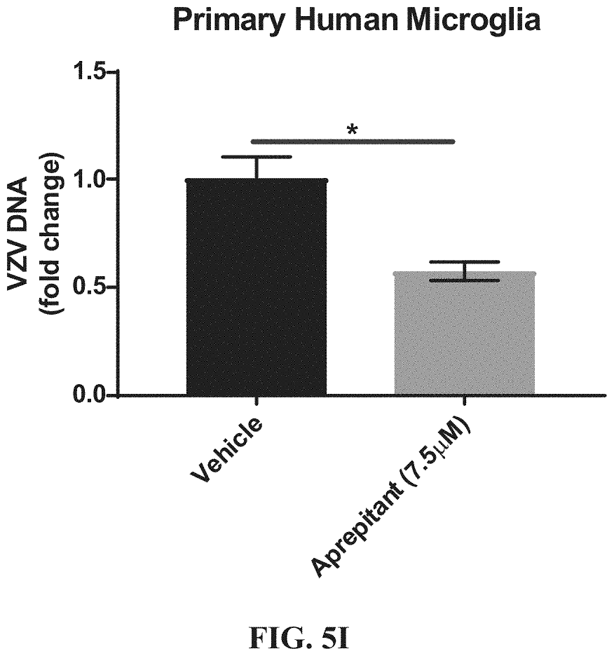

At 12 hours post-infection (HPI), quiescent spinal astrocytes were treated with vehicle or 10 μM aprepitant; the drug was reapplied at 24 and 48 HPI (timeline in FIG. 5A). At 12, 24, 48, and 72 HPI, cells were visualized by light microscopy in the untreated and treated cultures and harvested for DNA. As compared to untreated VZV-infected samples, aprepitant-treated VZV-infected cells had significantly reduced amounts of VZV DNA at 48 HPI (121±29.2 versus 10.5±3.8; p<0.01, mean±SD) and 72 HPI (452.9±122.9 versus 24.9±6.9; p<0.01, mean±SD) as quantified by qPCR (FIG. 5B). Corresponding light microscopy images demonstrated of a cytopathic effect at 48 and 72 HPI in VZV-infected/vehicle-only samples (FIG. 5C, arrows in upper panels), but not in aprepitant-treated samples (FIG. 5C, lower panels). Similarly, treatment with NK-1R antagonists aprepitant and rolapitant significantly reduced total VZV DNA in adult primary human perineurial cells (HPNCs), at 72 hours post-infection (HPI), when the cells were treated with either NK-1R antagonist at time of infection (FIG. 5D, left panel) as well as at 24 HPI (FIG. 5D, right panel). FIG. 5E demonstrates similar significantly reduced total VZV DNA by NK-1R antagonist treatment in primary adult human pancreatic islet cells (identified by insulin staining; “INS”) treated with aprepitant (7.5 μM) 6 days post-infection, compared to vehicle-treated islets. FIG. 5F demonstrates similar significantly reduced total VZV DNA by NK-1R antagonist treatment in adult primary human keratocytes treated with aprepitant (7.5 μM) 4 and 7-days post-infection (DPI), compared to vehicle-treated cells. FIG. 5G demonstrates a dose-dependent reduction in total VZV DNA in primary human brain vascular adventitial fibroblasts 72 hours post-infection following aprepitant treatment at 5 μM and 7.5 μM. FIG. 5I shows significantly reduced total VZV DNA by treatment with aprepitant (7.5 μM) in primary human microglia, compared to vehicle-treated cells.

The foregoing examples of the present invention have been presented for purposes of illustration and description. Furthermore, these examples are not intended to limit the invention to the form disclosed herein. Consequently, variations and modifications commensurate with the teachings of the description of the invention, and the skill or knowledge of the relevant art, are within the scope of the present invention. The specific embodiments described in the examples provided herein are intended to further explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1. A method of treating or preventing infection and spread of herpesviridae (HV) comprising providing a subject with protection against a HV infection or pathogenesis, by administering a sufficient amount of an NK-1R antagonist to provide the subject with protection against (SHV infection or pathogenesis.

2. (canceled)

3. A method of decreasing susceptibility or inhibiting HV reactivation from latency in a subject, comprising administering a sufficient amount of an NK-1R antagonist to decrease susceptibility or inhibit HV reactivation from latency in the subject.

4. The method of claim 1, wherein the NK-1R antagonist is aprepitant, rolapitant, fosaprepitant, lanepitant, befetupitant, or a combination thereof.

5. (canceled)

6. (canceled)

7. The method of claim 1, wherein the HV is an alpha-herpes virus and wherein the alpha herpes virus is herpes simplex virus-1 (HSV-1), herpes simplex virus-2 (HSV-2), or varicella zoster virus (VZV/HHV-3).

8. (canceled)

9. (canceled)

10. (canceled)

11. The method of claim 1, wherein the NK-1R antagonist prevents or inhibits a worsening or progression of HV infection or one or more symptoms or pathologies associated with HV infection, or pathogenesis or reactivation of HV from latency.

12. The method of claim 11, wherein the symptoms or pathologies treated or prevented include myelitis, myelopathy, neuropathy, pancreatitis, VZV-associated diabetic complications, vasculopathy, lesions, ulcers, canker sore, close sore, rash, boils, gingivostomatitis, herpes gladiatorum, eczema herpeticum, swollen lymph nodes, pneumonitis, pneumonia, hepatitis, meningitis, encephalitis, keratitis, genital herpes, esophagitis, hemiparesis, shingles, chicken pox, mononucleosis, chronic or acute pelvic inflammatory disease (PID), proctitis, colitis, and/or nerve damage.

13. (canceled)

14. The method of claim 1, wherein the NK-1R antagonist reduces or decreases HV titer, viral load, viral replication, viral proliferation or a viral protein, or inhibit or prevents increases in HV titer, viral load, viral replication, viral proliferation, or a viral protein.

15. (canceled)

16. The method of claim 1, wherein the subject is immunocompromised, or is a candidate for or has received an immunosuppressant treatment, or is a candidate for or has received a tissue or organ transplant.

17. The method of claim 1, further comprising administering to the subject an additional HV treatment, wherein the additional HV treatment comprises at least one of a protease inhibitor, a reverse transcriptase inhibitor, a virus fusion inhibitor, and a virus entry inhibitor.

18. (canceled)

19. The method of claim 1, wherein the HV infection is in spinal astrocytes in the subject and the pathogenesis is VZV myelitis and/or VZV myelopathy.

20. The method of claim 1, wherein the HV infection is in perineurial cells in the subject and the pathogenesis is VZV neuropathy.

21. The method of claim 1, wherein the HV infection is in pancreatic islet cells in the subject and the pathogenesis is VZV pancreatitis and/or VZV-associated diabetic complications.

22. The method of claim 1, wherein the HV infection is in keratocytes in the subject and the pathogenesis is VZV keratitis.

23. The method of claim 1, wherein the HV infection is in brain vascular adventitial fibroblasts and the pathogenesis is VZV vasculopathy.

24. The method of claim 3, wherein the NK-1R antagonist is aprepitant, rolapitant, fosaprepitant, lanepitant, befetupitant, or a combination thereof.

25. The method of claim 3, wherein the HV is an alpha-herpes virus and wherein the alpha herpes virus is herpes simplex virus-1 (HSV-1), herpes simplex virus-2 (HSV-2), or varicella zoster virus (VZV/HHV-3).

26. The method of claim 3, wherein the NK-1R antagonist prevents or inhibits a worsening or progression of HV infection or one or more symptoms or pathologies associated with HV infection, or pathogenesis or reactivation of HV from latency.

27. The method of claim 26, wherein the symptoms or pathologies treated or prevented include myelitis, myelopathy, neuropathy, pancreatitis, VZV-associated diabetic complications, vasculopathy, lesions, ulcers, canker sore, close sore, rash, boils, gingivostomatitis, herpes gladiatorum, eczema herpeticum, swollen lymph nodes, pneumonitis, pneumonia, hepatitis, meningitis, encephalitis, keratitis, genital herpes, esophagitis, hemiparesis, shingles, chicken pox, mononucleosis, chronic or acute pelvic inflammatory disease (PID), proctitis, colitis, and/or nerve damage.

28. The method of claim 3, wherein the NK-1R antagonist reduces or decreases HV titer, viral load, viral replication, viral proliferation or a viral protein, or inhibit or prevents increases in HV titer, viral load, viral replication, viral proliferation, or a viral protein.

29. The method of claim 3, wherein the subject is immunocompromised, or is a candidate for or has received an immunosuppressant treatment, or is a candidate for or has received a tissue or organ transplant.

30. The method of claim 3, further comprising administering to the subject an additional HV treatment, wherein the additional HV treatment comprises at least one of a protease inhibitor, a reverse transcriptase inhibitor, a virus fusion inhibitor, and a virus entry inhibitor.

31. The method of claim 3, wherein the subject has an HV infection, wherein the HV infection is in keratocytes in the subject and the pathogenesis is VZV keratitis.
